(19) United States
(12) Patent Application Publication
(10) Pub. No.: US 2007/0020317 A1
(43) Pub. Date: Jan. 25, 2007

(54) Negative ionic silica gel composition and the process for producing the same

(75) Inventor: Ricky Yeung, Central (HK)

Correspondence Address: Stephen E. Feldman, P.C., 7th FL, 12 East 41st Street, New York, NY, 10017, US

(73) Assignee: SAR Holdings International Limited

(21) Appl. No.: 11/490,002

(22) Filed: Jul. 20, 2006

(30) Foreign Application Priority Data
Jul. 22, 2005 (CN) 200510089524.3

(51) Int. Cl. A61K 9/70; A61K 33/00

(52) U.S. Cl. 424/443; 424/724

(57) ABSTRACT

The present invention relates to a negative ionic silica gel composition and the method of manufacturing the same. The composition includes negative ionic powder and silica gel. The negative ionic silica gel can be used to manufacture various suitable products or coat the surfaces of the products thereby adding the negative ions on them, thus purifying the environment and safeguarding human health.

### FIELD OF INVENTION

The present invention relates to a negative ionic silica gel composition, more particularly, the invention relates to a negative ionic silica gel composition which includes, in its most basic form, a negative ionic powder and silica gel. This invention also relates to a method of producing the composition and products formed therewith.

### BACKGROUND OF THE INVENTION

Negative ions are of great benefit to human health, natural life and biogeocenose, it is called as “Environment Police”, “Vitamin in Air”, or “Longevous Agent in Atmosphere.” The advantages of negative ions in human health care and environmental improvements are: purifying air, cleaning water, removing smog, removing bad smells, eliminating extra positive ions from electronic devices and/or equipments, improving lung functions, purifying blood, promoting blood circulation, increasing energy, and increasing appetite. Negative ions not only can ameliorate functions of respiratory system, circulatory system and nervous system, but also adjust and improve human metabolism and immunity, etc.

There are many products based on negative ion's benefits to human health care and environment improvements being developed, such as negative ionic underwear, negative ionic healthy sports socks, negative ionic water, negative ionic producer, negative ionic air-conditioners, negative ionic hair dryers, negative ionic refrigerator, etc.

Chinese patent application CN1542054A discloses a nanoscale negative ionic silica gel product and the method of producing the same; the nanoscale negative ionic silica gel comprises silica gel, nanoscale negative ionic powder, nanoscale argentums powder and magnetic material, wherein the silica gel is methyl vinyl silicone rubber, the nanoscale negative ionic powder is boron featured silicate with annular structured aluminum, sodium, iron and lithium. The method of this application has the steps of equably dispersing the nanoscale negative ionic powder into silica gel, then kneading and inpouring into module to produce the negative ionic product.

### SUMMARY OF THE INVENTION

The object of the present invention is to provide a negative ionic silica gel composition that is more convenient and efficient in providing negative ions. The composition can be either (1) added into silica gels that form various gel products, or (2) coated on any suitable products, thus bringing negative ions to almost any product.

One aspect of the present invention is to provide a negative ionic silica gel composition that includes, in its most basic form, a silica gel and negative ionic powder (NIP).

Another aspect of this invention is to provide a method of producing the negative ionic silica gel composition, which comprises the step of mixing the silica gel and NIP.

This invention also relates to a negative ionic product made by the negative ionic silica gel composition, and the method of producing the product.

### DETAILED DESCRIPTION OF THE INVENTION

Negative ionic powder (NIP):

Various Commercial available NIPs are used in many products such as paint, textile, plate, etc. Normally, suitable NIPs for silica gel are metal ore powders processed from natural ores. Natural ores are able to excite air thereby releasing a negative ion itself, it does not resort to any other outside power. The prime example of this ore-type is a well known named tourmaline.

The technology for processing the natural ore can be shattering, aftercuring, grinding, and drying the powder and/or removing the agglomerate among powder if needed. These technologies are already known in the art.

In the present invention, the preferred NIPs are ores selected from a group that includes, but not limited to, monazite, argil, heluguson and any combination of these. A more preferred NIP is a composite NIP that processed by various minerals intermixtures. The most preferred NIP in the present invention is obtained by processing the combination of monazite, argil and heluguson. The ratio of monazite, argil and heluguson in the combination can be 1: 1: 1. Such a composite NIP is produced by Kawashima Industrial Co. Ltd.

The present invention does not have a restriction on the granularity of NIP; it can be vary from few nanometers to few microns according to the process requirements.

Silica Gel:

The present invention does not have a restriction on the silica gel type. Suitable silica gels found in the art.are dimethylsiloxane polymer, methylvinylsiloxane polymer, methylstyrylsiloxane polymer and methyltrifluoropropylsiloxane polymer, among others gels not listed here. The silica gels could be used alone or in combination with other gels.

The preferred silica gel is siloxane elastomer which is obtained by a cross-linkage reaction of siloxane with cross linking agent. Preferred capable siloxane for cross linking is dimethylvinylsiloxane (VMQ) gel, the most preferred VMQ gel is with the polymerization degree between 7000-8000.

The cross linking agent can be a peroxide or metal platinum (Pt). The peroxide including benzoperoxide, 2,4-dichloro-benzoperoxide, dicumyl peroxide, di-t-butyl peroxide, p-I-chlorobutyryl peroxide, 2,5-dimethyl-2,5-bis-t-butyl hexaneperoxide, bis(t-butyl peroxy)peroxide, 2,5-dimethyl-2,5-bis(t-butyl peroxy)hexane, t-butyl cumyl peroxide.

The preferred cross linking agent is 2,5-dimethyl-2,5-bis-t-buty I hexaneperoxide.

More preferred cross linking agents are commercial available products named C-8, C-8A or C-8B manufactured by Shin-Etsu Silicone, Japan. The amount of the peroxide cross linking agent is about 0.3%-4% of the amount of other ingredients in weight.

The negative ionic silica gel of the present invention, the ratio of NIP and silica gel by weight is approximately 1:5˜3:2, preferred ratio is 1:2˜1:1, 1:1 being the most preferred.

The negative ionic silica gel composition can be used as additive to add into raw silica gel to manufacture needed products, thus to make them comprise negative ionic. The proportion of NIP in the products is about 5˜30% by weight, preferred 5˜20%, the most preferred being 5˜10%.

Or, the negative ionic silica gel composition can be made into a coating that covers a product.

The negative ionic silica gel composition can be produced by the following methods:

Pugging silica gel and NIP with appropriate proportion by weight in a pugging machine to make them fully mixed. There's no restriction on the time for pugging, so far as the silica gel is completely blended with the NIP, ordinarily, 0.5 to 1 hour is enough to achieve the result.

Other accessorial additives can also be added into the composition to make it has various purposes, if they do not affect the essential feature of the composition.

Mix the negative ionic silica gel composition together with appropriate amount of silica gel for manufacturing products, and add vulcanizing agent or other accessorial ingredients such as pigment, if needed, then mold the mixture in common way, and post-treat it by the ordinary way such as baking or purging to obtain the final products such as wristbands, necklaces, mattress, insoles, pillows, bandages, waistbands etc.

Or, mix the NIP together with liquid silica gel to get the negative ionic silica gel composition in paint, and then coat the composition on any product, and then post-treat the product, e.g. baking. The final products comprise a negative ionic such as negative ionic underwear, quilts, clothes and hats, blowing boards of air-conditioner, and water treatment devices.

The type of paint of negative ionic silica gel composition is more convenient in using and can be covered by any ordinary way, such as spraying, brushing or imprinting.

Below are Examples showing the Preferred Embodiments of the Invention

### EXAMPLE 1

Providing 5 shares of dimethylvinylsiloxane gel (by weight) and 5 shares of NIP (by weight), put these two components into a pugging machine and pugging them for 1 hour under normal pressure and temperature. The NIP used here is made by Kawashima Industrial Co. Ltd., and the granularity is about 5 μm. Vulcanize-pressing the negative ionic product obtained by pugging together with 2 shares of C-8 (by weight) and 100 shares of dimethylvinylsiloxane gel (by weight) under 165° C. for 10 minutes by normal compression molding way, then bake the component under 200° C. for 4 hours. Furthermore, clean the semi-product by boiling water for 4 hours, and then treating it by ultrasonic for 15 minutes to obtain the final product.

### EXAMPLE 2

Providing 10 shares of dimethylvinylsiloxane gel (by weight) and 10 shares of NIP (by weight), put these two components into a pugging machine and pugging them for 1 hour under normal pressure and temperature. The NIP used here is made by Kawashima Industrial Co. Ltd., and the granularity is about 5 μm. Vulcanize-pressing the negative ionic product obtained by pugging together with 0.5 share of C-8A (by weight) and 100 shares of dimethylvinylsiloxane gel (by weight) under 165° C. for 10 minutes by normal compression molding way, then bake the component under 200° C. for 4 hours. Furthermore, clean the semi product by boiling water for 4 hours, and then treating it by ultrasonic for 15 minutes to obtain the final product.

### EXAMPLE 3

Providing 25 shares of dimethylvinylsiloxane gel (by weight) and 25 shares of NIP (by weight), putting these two components into a pugging machine and pugging them for 1 hour under normal pressure and temperature. The NIP used here is made by Kawashima Industrial Co. Ltd., and the granularity is about 5 μm. Vulcanize-pressing the negative ionic product obtained by pugging together with 1 share of C-8B (by weight) and 100 shares of dimethylvinylsiloxane gel (by weight) under 165° C. for 10 minutes by normal compression molding way, then bake the component under 200° C. for 4 hours. Furthermore, clean the semi product by boiling water for 4 hours, and then treat it with ultrasonic for 15 minutes to obtain the final product.

### EXAMPLE 4

Mix 5 shares of dimethylvinyl silicone oil (by weight) and 5 shares of NIP (by weight) for 1 hour. The NIP used here is made by Kawashima Industrial Co. Ltd., and the granularity is about 5 μm. Mix the negative ionic product obtained above with 100 shares of dimethylvinylsiloxane gel (by weight) on the present of Pt, spray the mixture on the surface of a molded wristband, then bake it under 200° C. for 4 hours. Further, clean the achieved product by boiling water for 4 hours, and then treat it by ultrasonic for 15 minutes to obtain the final product.

Analyzing Negative Ionic Products:

Acquire a negative ionic silica gel paper in following of example 1. Put a measuring cup fill of water on the paper, the pH value of the water is about 7.2 assayed by pH Tester. Placing the water for 8 hours and assay the pH value of the water again, the result is about 8.3.

Although preferred embodiments have been depicted and described in detail herein, it will be apparent to those skillful technicians in the relevant art that various additions, substitutions, modifications and the like can be made without departing from the spirit of the invention and these are therefore considered to be within the scope of the invention.

What is claimed is:

1. A negative ionic silica gel composition comprising negative ionic powder and silica gel.

2. The composition as claimed in claim 1, wherein said silica gel is siloxane elastomer obtained by a cross-linkage reaction of siloxane with a cross-linking agent.

3. The composition as claimed in claim 1, wherein said silica gel is dimethylvinylsiloxane gel.

4. The composition as claimed in claim 3, wherein said silica gel has a polymerization degree of about 7000 to about 8000.

5. The composition as claimed in anyone of claims 1, wherein said negative ionic powder is a metal ore powder.

6. The composition as claimed in claim 5, wherein said negative ionic powder is selected from the group consisting of monazite, argil, heluguson and any combination of these.

7. The composition as claimed in claim 6, wherein said negative ionic powder is a composite negative ionic powder that is processed by various mineral intermixtures.

8. The composition as claimed in claim 7, wherein said composite negative ionic powder is processed by the combination of monazite, argil and heluguson.

9. The composition as claimed in claim 8, wherein the ratio of monazite, argil and heluguson is about 1:1:1 by weight.

10. The composition as claimed in anyone of claims 1, wherein the ratio of negative ionic powder and silica gel is from about 1:5 to about 3:2 by weight.

11. The composition as claimed in claim 10, wherein the ratio of negative ionic powder and silica gel is from about 1:2 to about 1:1 by weight.

12. The composition as claimed in claim 11, wherein the ratio of negative ionic powder and silica gel is about 1:1.

13. A method of producing a negative ionic silica gel composition, said method comprising the step of:
mixing a negative ionic powder with a silica gel.

14. The method as claimed in claim 13, wherein the ratio of negative ionic powder and silica gel is from about 1:5 to about 3:2 by weight.

15. The method as claimed in claim 14, wherein the ratio of negative ionic powder and silica gel is from about 1:2 to about 1:1 by weight.

16. The method as claimed in claim 15, wherein the ratio of negative ionic powder and silica gel is about 1:1.

17. A negative ionic paint comprising:
a silica gel and a composition, wherein said paint having a negative ionic powder with proportion of about 5˜30% by weight in the total amount of the paint.

18. The paint as claimed in claim 17, wherein said negative ionic powder is metal ore powder.

19. The paint as claimed in claim 18, wherein said negative ionic powder is selected from the group consisting of monazite, argil, heluguson and any combination of these.

20. The paint as claimed in claim 19, wherein said composite negative ionic powder is processed by the combination of monazite, argil and heluguson.

21. The paint as claimed in claim 20, wherein the ratio of monazite, argil and heluguson is about 1:1:1 by weight.

22. The paint as claimed in claim 17, wherein the proportion of negative ionic powder in the paint is about 5˜20% by weight.

23. The paint as claimed in claim 22, wherein the proportion of negative ionic powder in the paint is about 5˜10% by weight.

24. A method of producing negative ionic paint comprising the steps of:
1) producing a negative ionic silica gel composition; and
2) putting the mixture of step 1) into appropriate amount of liquid silica gel, and adding a metal pt together with discretional accessorial agents into the mixture, then pugging it into paint by ordinary way; said appropriate amount of silica gel is the amount that makes the proportion of negative ionic powder in the paint is about 5˜30% by weight.

25. The method as claimed in claim 24, wherein the proportion of negative ionic powder in the paint is about 5˜20% by weight.

26. The method as claimed in claim 25, wherein the proportion of negative ionic powder in the paint is about 5˜10% by weight.

27. A method of producing negative ionic product comprising the steps of:
1) producing a negative ionic silica gel composition; and
2) putting the mixture of step 1) into appropriate amount of liquid silica gel, and adding a vulcanizing agent together with discretional accessorial agents into the mixture, molding it by ordinary way, then post-treating it to obtain the final product; said appropriate amount of silica gel is the amount that makes the proportion of negative ionic powder in the paint is about 5-30% by weight.

28. The method as claimed in claim 27, wherein the proportion of negative ionic powder in the paint is about 5˜20% by weight.

29. The method as claimed in claim 28, wherein the proportion of negative ionic powder in the paint is about 5˜10% by weight.

30. The method as claimed in claim 27, wherein the product is wristband, necklace, mattress, insole, pillow, bandage or wristband.

31. The method as claimed in claim 27 using the negative ionic paint to coat the negative ionic paint onto a molded product.

32. The method of using as claimed in claim 31, wherein said product is negative ionic underwear, quilt, cloth, hat, blowing board for air-conditioner or water treatment device.
